(19) United States
(12) Patent Application Publication
(10) Pub. No.: US 2004/0219211 A1
(43) Pub. Date: Nov. 4, 2004

(54) Pharmeceutical formulation comprising a proton pump inhibitor and antacids

(76) Inventors: Bruno Criere, Gravigny (FR); Nourredine Nouri, Cannes (FR); Ake Pilbrant, Kungsbacka (SE); Pascal Suplie, Montaure (FR); Jean-Marc Zuccarelli, Antibes (FR)

Correspondence Address: WHITE & CASE LLP, PATENT DEPARTMENT, 1155 AVENUE OF THE AMERICAS, NEW YORK, NY, 10036, US

(21) Appl. No.: 10/484,064

(22) PCT Filed: Jul. 10, 2002
(86) PCT No.: PCT/SE02/01370

(30) Foreign Application Priority Data
Jul. 16, 2001 (EP) 01401896.4

(51) Int. Cl. A61K 9/26

(52) U.S. Cl. 424/469; 514/338

(57) ABSTRACT

The present invention deals with a multiparticulate tablet, which disintegrates in the mouth containing: i) a proton pump inhibiting agent, in particular of the benzimidazole type, in the form of enteric coated microgranules, which enteric coated granules are overcoated with at least one barrier coating, such as for instance a methacrylic copolymer-based protective film; ii) at least one antacid in the form of granules, for instance based on CaCO₃ and/or Mg(OH)₂ and/or Al(OH)₃; and, iii) a mixture of excipients comprising at least one disintegrating agent, one diluent agent, a lubricant, and optionally a swelling agent, a permeabilising agent, sweeteners, flavourings and colours. Furthermore, the present invention is directed to processes for the manufacture of the tablet and its use in the treatment of gastrointestinal disorders.

### FIELD OF THE INVENTION

The present invention is related to new oral pharmaceutical preparations especially for use in the prevention and treatment of gastrointestinal disorders. The present preparations comprise a combination of a proton pump inhibitor and an antacid agent in a tablet dosage form that disintegrates in the mouth.

Furthermore, the present invention refers to processes for the preparation of such a tablet and its use in the treatment of gastrointestinal disorders.

### BACKGROUND OF THE INVENTION AND PRIOR ART

Various methods and agents have been used to treat and/or eradicate gastrointestinal disorders. These include special diets, refraining from ingestion of certain foods, exercise, meditation, and administration of various pharmaceutical agents such as antacids, H₂ antagonists, and antimicrobials. One of the main treatments of today includes the class of pharmaceutical agents, referred to as proton pump inhibitors, that has been developed for treating gastrointestinal disorders. Proton pump inhibitors are agents which suppress gastric acid secretion by irreversible inhibition of the H⁺/K⁺-ATPase enzyme system in the parietal cell.

However, given the prevalence and incidence of gastrointestinal disorders, the difficulty in treating many patients suffering from such disorders, and the potential for resistance with antibiotic-containing regimens, a continuing need exists for safe and effective treatments, which are convenient, have good patient compliance and which provide individuals relief from their discomfort.

The administering of a proton pump inhibitor and an antacid rafting agent performed simultaneously but separately has been described in patent application WO 98/23272. The antacid rafting agent is a combination of an antacid agent with one alginate compound. The administering of 40 mg of omeprazole daily for about 28 days and the administering of one tablet of Gaviscon® four times a day for about 28 days, which delivers a total of 1280 mg of aluminium hydroxide and 320 milligrams of magnesium silicate per day, has been more precisely described. This treatment provides a therapy that shows a bad patient compliance due to the high number of daily doses. Moreover, further compliance problems arrive when proton pump inhibitor and antacid rafting agent are administered for different time periods and consist of different galenic formulations. Administration of two or even more different tablets to the patient is not convenient or satisfactory to achieve the most optimal results.

WO 97/25066 discloses an oral, multiple unit tableted dosage form comprising an acid susceptible proton pump inhibitor and one or more antacid agents or an alginate in a fixed combination formulation, wherein the proton pump inhibitor is in the form of individually enteric coating layered units. The units may also comprise an optional separating layer in between the proton pump inhibitor and the enteric coating. The antacid agent is for instance a mixture of magnesium hydroxide and calcium carbonate or a mixture of aluminium hydroxide and calcium carbonate.

The enteric coating layer covering the individual units of the said susceptible proton pump inhibitor has properties such that the compression of the units into a tablet does not significantly affect the acid resistance of the individually enteric coating layered units.

A tableted multiple unit effervescent dosage form has also been described in WO 97/25030. Enteric coating layered units containing the active substance is mixed with effervescent tablet constituents. The compression does not significantly affect the acid resistance of the enteric coating layered pellets, that may further be covered with one or more overcoating layers. Said overcoating enhances compressibility during tableting.

Oral disintegrable multiparticulate tablets have been already described in EP548356, EP1003484, WO00/27357 and WO00/51568, the content of which is hereby incorporated by reference. The active ingredient is in the form of coated microcrystals or coated microgranules.

Omeprazole and more generally proton pump inhibitors of the benzamidazole type must be protected with a gastro resistant polymer (enteric coating layer). Enteric films do not show high flexibility so that compression stress can yield rupturing of the film. It is therefore necessary to use a tableting technique that endorses the compression strain and maintains the acid resistance of the formulation after compression of the pellets. Such a formulation technology is described in WO 96/01623 hereby incorporated by reference. In the case of oral disintegradable multiparticulate tablets it has been found that it is also necessary to prevent degradation of the enteric coating film from penetration of saliva into the film. This provokes high stability problems. It has also been found that after disintegration of the tablet in the mouth and swallowing, the antacid agents make pH of the gastric contents rise to a pH value sufficient to provoke solubilisation of the enteric film coating. In order to solve the above-mentioned problems, the present invention provides a barrier layer to cover the enteric coating film.

### OUTLINE OF THE INVENTION

A first object of the invention is to provide a multiparticulate tablet, containing a proton pump inhibitor and an antacid agent, that disintegrates in the mouth and provides a good mouth feeling.

Another object of the present invention is to ensure the stability of the enteric coating film within the oral disintegradable tablet containing the antacid agent together with enteric coated proton pump inhibitor microgranules during storage.

It is also an object of the present invention to ensure integrity of the enteric film coating the proton pump inhibitor microgranules during use. The local pH in the antacid part of the tablet is around 9. A barrier coating is applied to protect the enteric coating from dissolution and/or disintegration in the mouth and/or stomach before the microgranules are transported into the small intestine. The tablet according to the present invention must also show satisfactory enteric properties of enteric microgranules, and provide a quick dissolution of the proton pump inhibitor in the small intestine.

The present invention particularly deals with a multiparticulate tablet, which disintegrates in the mouth containing:

i) a proton pump inhibiting agent, in particular of the benzimidazole type, in the form of enteric coating layered microgranules and which are overcoated with at least one barrier coating protecting the enteric coating from dissolution and/or disintegrating during the transport of the microgranules into the small intestine;

ii) at least one antacid in the form of granules, and;

iii) a mixture of excipients comprising at least one disintegrating agent, one diluent agent and, a lubricant,

Optionally, the multiparticulate tablet comprises a swelling agent, a permeabilising agent, sweeteners, flavourings, cooling agents and colours.

The term “proton pump inhibitor”, as used herein refers to any agent within the class of antisecretory compounds, which suppress gastric acid secretion by irreversible inhibition of the H⁺/K⁺ ATPase enzyme system at the secretory surface of the parietal cell.

These agents block the final step of acid production with regard to both basal and stimulated acid secretion irrespective of the stimulus. Proton pump inhibitors of the benzimidazole type are described in greater detail in Remington: The Science and Practice of Pharmacy, Vol. II, Nineteenth Edition, 892-3 (1995), incorporated herein by reference. Proton pump inhibitors are susceptible to degradation and/or transformation in acid reacting and neutral media and must therefore be protected from contact with acid gastric juice by an enteric coating layer.

Omeprazole; lansoprazole; pantoprazole; rabeprazole; leminoprazole; and mixtures thereof, are proton pump inhibitors, which are preferred for use in the present invention. The proton pump inhibitor may be used in the form of its racemate or a single enatiomer, in the non-salt form or in the form of an alkaline salt of the racemate or one of its single enantiomers. Omeprazole, in particular the magnesium salt thereof or the (S)-isomer of omeprazole in the form of a magnesium salt, are most preferred.

According to a preferred embodiment, the proton pump inhibiting agent is prepared in the form of enteric coating layered microgranules consisting of a core comprising the said agent optionally in mixture with an alkaline reacting compound. The core is covered by a separating layer and an enteric coating layer, and the enteric coated microgranules being overcoated with the barrier coating, such as for instance a methacrylic copolymer-based film.

The particle size distribution of the enteric coating layered microgranules is between 100 to 800 μm, preferably between 200 and 500 μm, most preferably around 500 μm. Moreover, the barrier coating is preferably a methacrylic copolymer-based film. This barrier film is preferably obtained from a coating liquid of particles of the copolymers of which at least 90% of the particles have a particle size of less than 315 μm. The prepared coating liquid is either water-based or prepared with organic solvents, preferably a water-based dispersion due to environmental concerns. This coating liquid should also be able to be sprayed with conventional spray layering equipment.

The methacrylic copolymer-based barrier coating preferably comprises a butyl methacrylate/(2-dimethylaminoethyl) methacrylate/methyl methacrylate(1:2:1) copolymer.

Eudragit® E-PO which is a pH-dependant polymer, is preferred for use as barrier coating. A barrier coating comprising Eudragit® E-PO can be made mechanically flexible and, when applied in increasing amounts to enteric coating layered proton pump inhibitor microgranules, provide a corresponding increase in the delayed release (dissolution) of the barrier coating. Different times for the delayed dissolution of the barrier coating in a medium of alkaline pH can thus be obtained while maintaining the properties of the enteric coating of the omeprazole microgranules, i.e. good acid resistance and rapid dissolution in the buffer stage testing at pH 6.8 of the USP monograph. Eudragit® E-PO is a methacrylate copolymer obtained from Eudragit® E 100 by milling, yielding a fine powder presentation. The barrier coating can also comprise a combination of methacrylic copolymers, as for example Eudragit® L 30 with Eudragit® FS 30 D.

Insoluble acrylic polymers, such as for example Eudragit® NE 30 D, Eudragit® RL30D, Eudragit® RS30D may also be used alone, in combination or in mixture with pH-dependant polymers to form an efficient barrier coating.

The amount of barrier coating is preferably between 5% and 60% of the weight of the enteric coating layered proton pump inhibitor microgranules.

The preferred qualitative formula based on Eudragit® E-PO contains enteric coated pellets equivalent to 20 mg omeprazole/tablet, Eudragit® E-PO as barrier coating polymer, dibutylsebacate as plasticiser of the barrier coating, sodium laurylsulfate as an additive for dispersion of E-PO in aqueous solvent and magnesium stearate as a lubricant and a mineral charge of coating film.

The unit amount of such compound is calculated in order to obtain the different relative amount of Eudragit®E-PO in the barrier- and enteric-coated omeprazole pellets:

10% as the lowest quantity to provide a minimum delayed release time of approximately 10 minutes,

30% to provide an intermediate delayed release time of approximately 30 minutes,

60% as maximum value for a 60 minutes delayed release time.

Optionally, the barrier coating further comprises an opacifying agent, preferably titanium dioxide.

An optional final polymeric coating, soluble in acidic condition, such as a hypromellose based film, is applied over the methacrylic copolymer-based barrier coating.

According to a preferred embodiment, the methacrylic copolymer-based barrier coating is obtained from a composition containing the following constituents:

Eudragit® E PO (methacrylic copolymer),

Dibutyl sebacate,

Sodium lauryl sulphate,

Magnesium stearate,

Titanium dioxide

Purified water.

The present invention comprises at least one antacid in the form of granules.

The term “antacid agent”, or “antacid(s)” as used herein, refers to any compound, which reacts with hydrochloric acid to form salt and water. Antacid agents are fully described in the following publications which are incorporated herein by reference in their entireties: G.B. 925,001, to Fielding et al., published May 1, 1963; and Remington: The Science and Practice of Pharmacy, Vol. II, Nineteenth Edition, 886-890 (1995).

Antacid agents useful herein include but are not limited to: aluminium carbonate, aluminium hydroxide, aluminium phosphate, aluminium hydroxy-carbonate, dihydroxy aluminium sodium carbonate, aluminium magnesium glycinate, dihydroxy aluminium amino acetate, dihydroxy aluminium aminoacetic acid, calcium carbonate, calcium phosphate, aluminium magnesium hydrated sulfates, magnesium aluminate, magnesium alumino silicates, magnesium carbonate, magnesium glycinate, magnesium hydroxide, magnesium oxide, magnesium trisilicate, sucrafalte, sodium bicarbonate, and mixtures thereof.

The classical powder grades of antacid agents show bad tableting properties, and bad organoleptic properties especially regarding mouth feeling and taste. Therefore, the antacid agent is preferably used in the form of granules. Advantageously, the antacid is obtained by dry granulation of CaCO₃ and/or Mg(OH)₂ and/or Al(OH)₃ with mannitol, followed by wet granulation using a solution of xylitol and/or sorbitol. Antacid granules may optionally include a disintegrating agent and/or a permeabilisation agent.

Advantageously, the antacid granules according to the invention present particle size distribution between 150 μm and 710 μm, preferably between 355 μm and 710 μm, such that at least 50%, preferably at least 70% of the granules have a particle size ranging between 150 and 710 μm and less than 20% of the granules have a particle size less than 150 μm. The particle sizes are measured according to conventional methods, preferably by sieving.

The tablet of the invention also comprises a mixture of excipients.

The diluent agent may be selected from water-soluble and/or water-insoluble tabletting filler. The water-soluble diluent agent is constituted from a polyol of less than 13 carbon atoms, in the form of directly compressible material (the mean particle size being between 100 and 500 microns), in the form of a powder (the mean particle size being less than 100 microns) or a mixture thereof. The polyol is preferably chosen from the group comprising of mannitol, xylitol, sorbitol and maltitol. The water-insoluble diluent agent is a cellulosic derivative preferably microcrystalline cellulose.

The disintegrating agent is chosen from the group consisting of crosslinked sodium carboxymethylcellulose, crospovidone and their mixtures. A part of the disintegrating agent is advantagenously used for the preparation of antacid granules.

The lubricant agent is chosen from the group consisting of magnesium stearate, sodium stearylfumarate, stearic acid, Macrogol 6000 and their mixtures. A part of the lubricant is used as an internal solid lubricant, another part is advantageously sprayed over the outer surface of the tablet.

The swelling agent is chosen from the group consisting of starch, modified starch or microcrystalline cellulose.

The permeabilising agent is chosen from the group consisting of silica having a high affinity with aqueous solvents, such as Syloïd®, maltodextrines, beta-cyclodextrines and their mixtures. The permeabilising agent enables creation of a hydrophilic network that enhances the penetration of the saliva and the disintegration of the tablet. A part of permeabilising agent is advantageously used for the preparation of antacid granules.

The sweetener can be chosen in the group consisting of aspartame, potassium acesulfame, sodium saccharinate, dihydrochalcone neohesperidine and their mixtures.

The flavouring is advantageously chosen to give a combination of fast onset and long-lasting sweet taste and get a “round feeling” in the mouth with different texturers or additives.

A combination of potassium acesulfame with aspartame is particularly preferred as a sweetener agent.

Cooling agents can also be added in order to improve the mouth feeling and provide a synergy with flavours and sweetness.

According to a preferred embodiment, the tablet has the following composition:

i) Barrier coated omeprazole microgranules

Enteric coating layered omeprazole magnesium microgranules

Eudragit® E PO (methacrylic copolymer)

Dibutyl sebacate

Sodium lauryl sulphate

Magnesium stearate

Purified water

and optionally

Titanium dioxide

Hypromellose

Talcum

ii) Antacid granules

CaCO₃

Mg(OH)₂

Mannitol

Sorbitol

Purified water

and optionally

Crospovidone

Silica

iii) Excipients for formulation of the tablet

Mannitol or microcrystalline cellulose

Crospovidone or croscarmellose

Aspartame

Flavourings

Silica

Magnesium stearate

Water is used as solvent and removed during the coating and the granulation processes.

In one aspect of the invention, the tablet of the invention is an orodispersible multiparticulate tablet that disintegrates in contact with the saliva, without chewing, in less than 60 seconds, preferably in less than 40 seconds.

According to one preferred embodiment, the orodispersible tablet has the following composition:

i) Barrier coated omeprazole microgranules

Enteric coating layered omeprazole magnesium microgranules

Eudragit® E PO (methacrylic copolymer)

Dibutyl sebacate

Sodium lauryl sulphate

Magnesium stearate

Purified water

and optionally

Titanium dioxide

Hypromellose

Talcum

ii) Antacid granules

CaCO₃

Mg(OH)₂

Mannitol

Sorbitol

Purified water

and optionally

Crospovidone

Silica

iii) Excipients for formulation of the tablet

Mannitol

Crospovidone

Aspartame

Flavourings

Silica

Magnesium stearate

and optionally

Cooling agents

According to another preferred embodiment, the orodispersible tablet has the following composition:

i) Barrier coated omeprazole microgranules

Enteric coating layered omeprazole magnesium microgranules

Eudragit® E PO (methacrylic copolymer)

Dibutyl sebacate

Sodium lauryl sulphate

Magnesium stearate

Purified water

and optionally

Titanium dioxide

Hypromellose

Talcum

ii) Antacid granules

CaCO₃

Mg(OH)₂

Mannitol

Sorbitol

Purified water

and optionally

Crospovidone

Silica

iii) Excipients for formulation of the tablet

Microcrystalline cellulose

Crospovidone

Aspartame

Flavourings

Silica

Magnesium stearate

and optionally

Cooling agents

In another aspect of the invention, the invention is a chewable multiparticulate tablet. According to a preferred embodiment, the chewable tablet has the following composition:

i) Barrier coated omeprazole microgranules

Enteric coating layered omeprazole magnesium microgranules

Eudragit® E PO(methacrylic copolymer)

Dibutyl sebacate

Sodium lauryl sulphate

Magnesium stearate

Purified water

and optionally

Titanium dioxide

Hypromellose

Talcum

ii) Antacid granules

CaCO₃

Mg(OH)₂

Mannitol

Sorbitol

Purified water

and optionally

Crospovidone

Silica

iii) Excipients for formulation of the tablet

Microcrystalline cellulose

Croscarmellose

Aspartame

Flavourings

Silica

Magnesium stearate

and optionally

Cooling agents

According to a most preferred embodiment, the tablet of the invention, either orodispersible or chewable, has the following composition:

i) Barrier coated omeprazole microgranules
Enteric coating layered omeprazole microgranules
ca 100 mg/equivalent to 20 mg of omeprazole

| | | |
|---|---|---|
| Eudragit ® E PO | 10-60 | mg |
| Dibutyl sebacate | 1-10 | mg |
| Sodium lauryl sulphate | 0.5-5 | mg |
| Magnesium stearate | 2.5-15 | mg |

| | |
|---|---|
| Purified water | — |

ii) Antacid granules

| | | |
|---|---|---|
| CaCO₃ | 350-900 | mg |
| Mg(OH)₂ | 100-250 | mg |
| Mannitol | 70-330 | mg |
| Sorbitol | 30-90 | mg |
| Crospovidone | 0-50 | mg |
| Silica | 0-10 | mg |

| | |
|---|---|
| Purified water | — |

iii) Excipients for formulation of the tablet

| | | |
|---|---|---|
| Diluent agent | 200-600 | mg |
| Disintegrating agent | 50-300 | mg |
| Aspartame | 10-40 | mg |
| Flavourings | 10-30 | mg |
| Silica | 5-15 | mg |
| Magnesium stearate | 5-30 | mg |

Water is used as solvent and removed during the coating and the granulation processes.

The tablet according to the present invention preferably shows an acid binding capacity higher than 10 mEq/tablet and after administration to patients a rapid initial rise in gastric pH. Preferably the acid binding capacity is between 10 and 25 mEq/tablet. The enteric coating of the proton pump inhibitor microgranules complies with the requirements of the USP for enteric coated articles. The release of the proton pump inhibitor in the buffer stage testing (pH 6.8) shows not less than 80% released in 30 minutes. Furthermore, the tablet is preferable round with a diameter of less than 20 mm. Alternatively, the tablet may be oval-shaped.

The tablet according to the invention, has a hardness of not less than 15 N, preferably between 20 to 70 N, when measured with the test method of the European Pharmacopeia (2.9.8).

The present invention also refers to the use of a tablet as described above for the manufacture of a medicament for the treatment of gastrointestinal disorders.

The term “gastrointestinal disorder”, as used herein, encompasses any infection, disease or other disorder(s) of the upper gastrointestinal tract. Such disorders include, for example, heartburn; sour stomach; acid ingestion; upset stomach and/or pain associated with heartburn, sour stomach and acid ingestion; bloating; fullness; dyspepsia; hiatus hernia; esophagitis; nocturnal heartburn; erosive esophagitis; disorders not manifested by the presence of ulcerations in the gastric mucosa, including chronic active or atrophic gastritis, Zollinger-Ellison syndrome; non-ulcer dyspepsia, esophageal reflux disease and gastric motility disorders; peptic ulcer disease, i.e., pre-pyloric, marginal, and/or gastric, duodenal ulcers; and combinations thereof. Preferred for treatment by the present invention includes heartburn with and without stomach pain, dyspepsia, esophagitis, chronic active or atrophic gastritis and esophageal reflux disease.

The tablet is administered one to several times a day, preferably once or twice daily. The typical daily dose of the active substances varies and will depend on various factors such as the individual requirements of the patients and disease. In general, each tablet will comprise 10-80 mg of the proton pump inhibitor and 200-1500 mg of the antacid agent. Preferably, each tablet will comprise 10-40 mg of the proton pump inhibitor and 300-1000 mg of the antacid agents.

The invention is illustrated more in detail in the following examples.

### EXAMPLE 1

Tests of formulations with and without a barrier coating layer

Stability tests have been performed on the following samples:

Multiple unit tablets containing enteric coated pellets of omeprazole magnesium without any barrier coating,

Multiple unit tablets containing enteric coated pellets of omeprazole magnesium protected with a barrier coating of Eudragit® E-PO (methacrylic copolymer),

Multiple unit tablets containing enteric coated pellets of omeprazole magnesium barrier coated with Eudragit® L30 D and FS 30D.

These stability tests have been performed in aluminium/aluminium cold formed blisters in classical I.C.H. conditions (25° C./60% RH-30 ° C./60% RH-40° C./75% RH).

Results

Enteric coated omeprazole pellets without any barrier coating show an unsatisfactory enteric resistance, justifying the necessity of a barrier coating.

The stability of omeprazole in these preliminary tablets is satisfactory.

### EXAMPLE 2

To promote an acid binding capacity ≧10 mEq/tablet and to allow for good physical properties of the tablet (tableting behaviour, organoleptic properties and short disintegrating time), different formulations of the antacid agent have been explored. Granulation of the antacid compounds is preferred. Simple granulation, or granulation followed by a light coating phase can be performed in order to obtain a better taste and physical behaviour of the granules.

Furthermore, introduction of a filler allows for good taste and physical behaviour in the dry mix of antacids. Wetting and granulating with different aqueous binder solutions may further strengthen these characteristics. The best results were obtained by combining 12% mannitol in dry mix followed by granulation with xylitol or sorbitol solution.

The most preferred antacid formulation or a multiple thereof is the following:

| Components | Unit formula (mg) | Percent formula (%) |
|---|---|---|
| CaCO₃ | 350 | 63.6 |
| Mg(OH)₂ | 100 | 18.2 |
| Mannitol | 66.7 | 12.1 |
| Sorbitol | 33.3 | 6.1 |
| Total weight | 550 | 100 |

Another preferred composition comprises omeprazole magnesium in an amount corresponding to 20 mg omeprazole, 770 mg CaCO₃ and 220 mg Mg(OH)₂.

| Components | Unit formula (mg) | Percent formula (%) |
|---|---|---|
| CaCO₃ | 770 | 57.0 |
| Mg(OH)₂ | 220 | 16.3 |
| Mannitol | 293 | 21.8 |
| Sorbitol | 64 | 4.9 |
| Total weight | 1347 | 100 |

### EXAMPLE 3

The following formulation was prepared

| Components | Unit formula (mg) | Percent formula (%) |
|---|---|---|
| Barrier coated E.C.O.P. | Providing 20 mg | depending on |
| | omeprazole | amount of |
| | | coating |
| Antacids granulate | 550 mg | 39.3 |
| Mannitol | q.s. for tablet | |
| | depending on quantity of | |
| | barrier coated E.C.O.P. | |
| Crospovidone | 210 | 15 |
| Aspartame | 28 | 2 |
| Flavour | 11.5 | 0.82 |
| Silica | 7 | 0.5 |
| Magnesium stearate | 14 | 1 |
| Total weight | 1400 | 100 |

E.C.O.P. = enteric coated microgranules comprising omeprazole magnesium.

With a specific bi-convex shape, the 17 mm round tablets obtained are satisfactory regarding their fast dispersible characteristics in the mouth:

disintegrating time in mouth between 25 to 35 seconds,

no chalky taste nor granular mouth feeling,

good flavouring profile with a pleasant light cooling effect in the mouth.

### EXAMPLE 4

The following batches were prepared according to the formulae

| | 10% EPO | 30% EPO | 60% EPO |
|---|---|---|---|
| Components | (mg) | (mg) | (mg) |
| Barrier coated E.C.O.P. | | | |
| E.C.O.P. (4) | 100 | 100 | 100 |
| Equivalent to omeprazole (1) | (20) | (20) | (20) |
| Eudragit E-PO | 10 | 30 | 60 |
| Dibutylsebacate | 1.5 | 4.5 | 9.0 |
| Na laurylsulfate | 0.75 | 2.25 | 4.5 |
| Magnesium stearate | 2.5 | 7.5 | 15.0 |
| Purified water (2) | — | — | — |
| Total barrier coated E.C.O.P. | 114.75 | 144.25 | 188.5 |
| Antacids granules | | | |
| CaCO₃ | 350 | 350 | 350 |
| Mg(OH)₂ | 100 | 100 | 100 |
| Mannitol | 66.67 | 66.67 | 66.67 |
| Sorbitol | 33.33 | 33.33 | 33.33 |
| Purified water (2) | — | — | — |
| Total antacids granules | 550 | 550 | 550 |
| Tableting formula | | | |
| Mannitol (3) | 464.75 | 435.25 | 391 |
| Crospovidone | 210 | 210 | 210 |
| Aspartame | 28 | 28 | 28 |
| Flavour | 11.5 | 11.5 | 11.5 |
| Silica | 7 | 7 | 7 |
| Magnesium stearate | 14 | 14 | 14 |
| Total unit weight of tablet | 1400 | 1400 | 1400 |

(1): for a theoretical content in Omeprazole of E.C.O.P. of 20%
(2): water used as a solvent, eliminated during coating and granulation processes
(3): amount of mannitol adjusted to keep the unit weight of tablet to 1400 mg
(4): E.C.O.P.: Enteric Coated Pellets comprising omeprazole magnesium

Process for preparing the above formulae:

Enteric Coated Omeprazole Pellets (E.C.O.P). Pellets comprising omeprazole magnesium were prepared according to WO 96/01623, hereby incorporated by reference. The pellets were prepared in accordance with example 2 of WO 96/01623.

Barrier coating of the enteric coated omeprazole pellets.

2000 g Enteric coated omeprazole pellets were coated in a fluidised bed After coating the product was dried in the fluidised bed.

Granulation of antacids

Batch size 1.650 kg equivalent to 3000 units 350+100 mg dosed.

Dry pre-mix of antacids+mannitol in a rotary mixer granulator.

Wetting of the dry mix with a sorbitol aqueous solution.

Granulation after the end of wetting.

Transfer of the wet mass in a fluidised bed and drying.

Tableting

Mixing of barrier coated omeprazole pellets, antacid granules, and tablet excipients in a cubic mixer.

Tableting on a rotary laboratory machine equipped with 3 punches of specific shape and 17 mm diameter adapted to the 1400 mg unit weight.

Rotation speed 25 rpm.

Packaging operations

Performed in aluminium/aluminium cold formed blisters with embossing of the batch number.

Results

| | BATCH | | |
|---|---|---|---|
| | 10% EPO | 30% EPO | 60% EPO |
| Average weight | 1407 mg | 1400 mg | 1405 mg |
| Average thickness | 5.7 mm | 5.7 mm | 5.7 mm |
| Resistance to crushing | | | |
| Average | 31 N | 26 N | 26 N |
| Friability | 2.9% | 5.4% (2) | 3.3% |
| Disintegration time (in mouth) | 31 s | 29 s | 27 s |
| Acid resistance | 5.6% dissolved | 2.3% dissolved | 8.8% dissolved |
| (after 5 min in pH 6.8) | | | |
| Dissolution in pH 6.8 | 92.3% in 30 min | 90.8% in 30 min | 89.8% in 30 min |
| (after acid resistance stage) | | | |
| Barrier coating evaluation (in pH 6.8) | 2.1% in 10 min | 4.5% in 30 min | 4.9% in 60 min |
| Acid-neutralizing capacity | 10.0 mEq/tab | 10.3 mEq/tab | 10.8 mEq/tab |
| Omeprazole assay | 20.3 mg | 19.8 mg | 19.7 mg |
| | (101.5% theory) | (99.9% theory) | (98.5% theory) |

Total acid binding capacity (acid neutralising capacity) determined according to the USP 24 method. All results comply with the expected specification, i.e. value ≧10 mEq/tablet.

### EXAMPLE 5

The following formulations with the unit formulas below were prepared

| Components | Orodispersible tablet (mg) | Chewable tablet |
|---|---|---|
| Barrier coated | Providing 20 mg omeprazole | |
| E.C.O.P. | (quantity depending on coating factor) | |
| Antacids granulate | 1347 mg | |
| Microcrystalline | q.s. for tablet | |
| cellulose | depending on quantity of barrier | |
| | coated E.C.O.P. | |
| Crospovidone | 160 | 0/ |
| Croscarmellose | 0/ | 60 |
| Aspartame | 16.8 | |
| Acesulfame K | 11.2 | |
| Flavour | 16.4 | |
| Cooling agents | 1.2 | |
| Silica | 10 | |
| Magnesium stearate | 20 | |
| Total weight | 2000 | 2000 |

The following formulation was prepared

E.C.O.P.=enteric coated microgranules comprising omeprazole magnesium.

With a flat shape, the 18 mm round tablets obtained are satisfactory regarding their fast dispersible characteristics in the mouth, with and without chewing, respectively:

acceptable granular mouth feeling,

tablet unit weight and size acceptable for disintegration in mouth.

### EXAMPLE 6

The following batches were prepared according to the following formulae

| | 10% EPO | 30% EPO | 60% EPO |
|---|---|---|---|
| Components | (mg) | (mg) | (mg) |
| Barrier coated E.C.O.P. | | | |
| E.C.O.P. (4) | 100 | 100 | 100 |
| Equivalent to omeprazole (1) | (20) | (20) | (20) |
| Eudragit E-PO | 10 | 30 | 60 |
| Dibutylsebacate | 1.5 | 4.5 | 9.0 |
| Na laurylsulfate | 0.75 | 2.25 | 4.5 |
| Magnesium stearate | 2.5 | 7.5 | 15.0 |
| Titanium oxide | 4.0 | 4.0 | 4.0 |
| Hypromellose | 3.6 | 3.6 | 3.6 |
| Talcum | 0.89 | 0.89 | 0.89 |
| Purified water (2) | — | — | — |
| Total barrier coated E.C.O.P. | 123 | 154 | 200 |
| Antacids granules | | | |
| CaCO₃ | 770 | 770 | 770 |
| Mg(OH)₂ | 220 | 220293 | 220 |
| Mannitol | 293 | 64 | 293 |
| Sorbitol | 64 | — | 64 |
| Purified water (2) | — | 1347 | — |
| Total antacids granules | 1347 | | 1347 |
| Tableting formula | | | |
| Microcrystalline cellulose (3) | 29460 | 435 | 391 |
| Croscarmellose | 16.8 | 60 | 60 |
| Aspartame | 11.5 | 16.8 | 16.8 |
| Acesulfame K | 16.4 | 11.5 | 11.5 |
| Flavour | 1.2 | 16.4 | 16.4 |
| Cooling agent | 10 | 1.2 | 1.2 |
| Silica | 20 | 10 | 10 |
| Magnesium stearate | | 20 | 20 |
| Total unit weight of tablet | 2000 | 2000 | 2000 |

(1): for a theoretical content in Omeprazole of E.C.O.P. of 20%
(2): water used as a solvent, eliminated during coating and granulation processes
(3): amount of microcrystalline cellulose adapted in function of the real content of omeprazole of E.C.O.P in order to adjust the unit weight of 2000 mg
(4):: E.C.O.P.: Enteric Coated Pellets comprising omeprazole magnesium

Process for preparing the above formulae:

step 1: Enteric Coated Omeprazole Pellets (E.C.O.P) preparation.

Pellets comprising omeprazole magnesium were prepared according to WO 96/01623, hereby incorporated by reference. The pellets were prepared in accordance with example 2 of WO 96/01623.

step 2: barrier coating of the enteric coated omeprazole pellets.

1000 g Enteric coated omeprazole pellets were coated in a fluidised bed After coating the product was dried in the fluidised bed.

step 3: granulation of antacids

Batch size 2,450 kg equivalent to 1800 units 770+220 mg dosed.

Dry pre-mix of antacids+mannitol in a rotary mixer granulator.

Wetting of the dry mix with a sorbitol aqueous solution.

Granulation after the end of wetting.

Transfer of the wet mass in a fluidised bed and drying.

step 4: tabletting

Mixing of barrier coated omeprazole pellets, antacid granules, and tablet excipients in a cubic mixer.

Tableting on a rotary laboratory machine equipped with 3 punches of specific shape and 18 mm diameter adapted to the 2000 mg unit weight.

Rotation speed 25 rpm.

Packaging operations

Performed in aluminium/aluminium cold formed blisters with embossing of the batch number.

Results

| | BATCH | | |
|---|---|---|---|
| | 10% EPO | 30% EPO | 60% EPO |
| Average weight | 1999 mg | 2016 mg | 1984 mg |
| Average thickness | 5.5 mm | 5.6 mm | 5.6 mm |
| Resistance to crushing Average | 64 N | 54 N | 54 N |
| Friability | 0.7% | 1% | 0.8% |
| Disintegration time (in mouth) | 55 s | 50 s | 50 s |
| Acid resistance | 11% dissolved | 17% dissolved | 8% dissolved |
| (after 5 min in pH 6.8) | | | |
| Dissolution in pH 6.8 | 81% in 30 min | 79% in 30 min | 90% in 30 min |
| (after acid resistance stage) | | | |
| Barrier coating evaluation (in pH 6.8) | 3% in 10 min | 1% in 30 min | 4% in 30 min |
| Acid-neutralizing capacity | 22.0 mEq/tab | 23 mEq/ tab | 22 mEq/tab |
| Omeprazole assay | 20.3 mg | 19.9 mg | 19.6 mg |
| | (101.3% theory) | (99.4% theory) | (97.8% theory) |

Total acid binding capacity (acid neutralising capacity) determined according to the USP 24 method. All results comply with the expected specification, i.e. value ≧10 mEq/tablet.

### EXAMPLE 7

Tablets containing barrier coated E.C.O.P equivalent to 10 mg of omeprazole and antacid granules equivalent to 495 mg of antacids and halves of the amounts of all other ingredients were prepared following steps 1 to 3 of the process described in example 6.

step 4: tableting

Mixing of barrier coated omeprazole pellets, antacid granules, and tablet excipients in a cubic mixer.

Tableting on a rotary laboratory machine equipped with 3 punches of specific shape and 14 mm diameter adapted to the 1000 mg unit weight.

Rotation speed 25 rpm.

### Analytical Methods Used in the Present Examples

1. Release of Omeprazole

Several tests were performed to follow release of omeprazole from formulations: ECOP, protected ECOP and Flashtab®.

1.1. Acid Resistance after 5 Min Dispersion in pH 6.8

| | |
|---|---|
| Apparatus | 2 (paddle) |
| Rotation | 100 ± 4 rpm |
| Medium | 10 mL of pH 6.8 buffer for 5 min and addition of |
| | 740 mL of 0.1 N hydrochloric acid |
| | pH 6.8 buffer: 75 mL of 0.1 N hydrochloric acid, 25 mL of |
| | tribasic sodium phosphate 0.2 M, adjustment to pH 6.8 with |
| | 2 N hydrochloric acid; |
| | 5 min: simulating the transit time in and just after mouth; |
| Temperature | 37 ± 0.5° C. |
| Sample | 1 tablet or a quantity of in process material equivalent |
| | to 20 mg of omeprazole |
| Time | 2 hours after hydrochloric acid addition salt |
| | (total: 2 h 05 min) |
| Analysis | by the HPLC method described for Assay on the insoluble |
| | recovered by medium filtration |

1.2. Dissolution in Buffer pH 6.8 after Acid Resistance Stage

| | |
|---|---|
| Apparatus | 2 (paddle) |
| Rotation | 100 ± 4 rpm |
| Medium | 10 mL of pH 6.8 buffer (as above) for 5 min addition of |
| | 740 mL of 0.1 N hydrochloric acid, opereation for 2 hours |
| | and addition of 250 mL of tribasic sodium phosphate 0.2 M |
| Temperature | 37 ± 0.5° C. |
| Sample | 1 tablet or a quantity of in process material equivalent to |
| | 20 mg of omeprazole |
| Time | 30 min after tribasic sodium phosphate addition |
| | (total: 2 h 35 min) |
| Analysis | by the HPLC method described for Assay on an aliquot |
| | of the medium |

1.3. Barrier-Coating Evaluation in pH 6.8

| | |
|---|---|
| Apparatus | 2 (paddle) |
| Rotation | 100 ± 4 rpm |
| Medium | 500 mL of pH 6.8 buffer(as above) |
| Temperature | 37 ± 0.5° C. |
| Sample | 1 tablet or a quantity of in process material equivalent to |
| | 20 mg of omeprazole |
| Time | 10, 30 and 60 min |
| Analysis | UV spectrophotometric on-line detection at 300 nm |

2.1 Acid-Neutralizing Capacity (Example 4)

The method is described in USP 24, page 1863 <301 > for nonchewable tablets without addition of alcohol.

2.2 Acid-Neutralizing Capacity (Example 6)

Determined at a constant pH using a Karl Fischer titrator.

Determination of acid consumed after 10 minutes and 30 minutes.

Equivalent of one tablet in a beaker with 5 ml of acified water (pH 3.0), placed in a thermostated water bath at 37° C., 15 minutes.

Addition of 30 ml of acified water at 37° C.

Titration with HCl 1M, and titrator arranged as a pH-stat at 3.0.

3. Omeprazole Assay

| | |
|---|---|
| An HPLC method: | conditions described below. |
| Column | C18 − 250 × 4.6 mm − 5μ with a 3 mm pre-column |
| Column temperature | 40° C. |
| Mobile phase | mixture of acetonitrile, 2% v/v triethanolamine |
| | solution (50:50) adjusted to pH 8.50 ± 0.05 with |
| | phosphoric acid |
| Flow rate | 0.7 mL:min |
| Injection | 20 μL |
| Detection | 300 nm |
| Extraction solvent | mixture of acetonitrile, 2% v/v triethanolamine |
| | solution (50:50) |
| Concentration level | 0.01 mg/mL |

1. A multiparticulate tablet which disintegrates in the mouth of a patient, wherein the multiparticulate tablet comprises:
i) a proton pump inhibiting agent in the form of microgranules layered with an enteric coating, wherein the enteric coating layered microgranules are overcoated with at least one barrier coating that protects the enteric coating from dissolution and/or disintegration during the transport of the microgranules into the small intestine;
ii) at least one antacid agent in the form of granules and;
iii) a mixture of excipients comprising at least one disintegrating agent, one diluent agent, and a lubricant.

2. The tablet according to claim 1, wherein the proton pump inhibiting agent is omeprazole, an alkaline salt of omeprazole, a single enantiomer of omeprazole or an alkaline salt of the single enantiomer.

3. The tablet according to claim 2, wherein the proton pump inhibiting agent is the (S)-isomer of omeprazole or the alkaline salt thereof.

4. The tablet according to claim 2, wherein the proton pump inhibiting agent is a magnesium salt of either omeprazole or the (S)-isomer of omeprazole.

5. The tablet according to claim 1, wherein the proton pump inhibiting agent is a compound selected from the group consisting of lansoprazole, pantoprazole, rabeprazole and leminoprazole, an alkaline salt of the compound, a single enantiomer of the compound or an alkaline salt of the single enantiomer.

6. The tablet according to claim 1, wherein the enteric coating layered microgranules comprise the following:
i. a core comprising the proton pump inhibiting agent, an alkaline salt of the proton pump inhibiting agent, a single enantiomer of the proton pump inhibiting agent or an alkaline salt of the single enantiomer, optionally combined with an alkaline reacting compound;
ii. a separating layer covering the core; and
iii. an enteric coating layer,
wherein the separating layer separates the core from the enteric coating layer.

7. The tablet according to claim 1, wherein the particle size of the enteric coating layered microgranules is in the range between 100 and 800 microns.

8. The tablet according to claim 1, wherein the barrier coating is a methacrylic copolymer-based film.

9. The tablet according to claim 8, wherein the barrier coating is prepared from particles of a methacrylic copolymer, and wherein at least 90% of the particles of the copolymer have a particle size less than 315 μm.

10. The tablet according to claim 8, wherein the barrier coating is prepared from a methacrylic copolymer in a water based dispersion.

11. The tablet according to claim 8, wherein the barrier coating comprises a butyl methacrylate/(2-dimethylaminoethyl) methacrylate/methyl methacrylate(1:2:1) copolymer.

12. The tablet according to claim 8, wherein the 5 to 60% weight of the enteric coating layered microgranules is comprised of the barrier coating.

13. The tablet according to claim 8, wherein the barrier coating comprises:
i Eudragit® E PO (methacrylic copolymer),
ii. dibutyl sebacate,
iii. sodium lauryl sulphate,
iv. magnesium stearate,
v. titanium dioxide
vi. purified water.

14. The tablet according to claim 1, wherein the antacid is based on CaCO₃ and/or Mg(OH)₂ and/or Al(OH)₃.

15. The tablet according to claim 1, wherein the antacid granules comprise a disintegrating agent and/or a permeabilising agent.

16. The tablet according to claim 1, wherein at least 50% of the antacid granules have a particle size ranging between 150 and 710 μm and less than 20% of the antacid granules have a particle size less than 150 μm.

17. The tablet according to claim 1, wherein the diluent agent is a polyol of less than 13 carbon atoms or a cellulosic derivative.

18. The tablet according to claim 17, wherein the polyol is selected from the group consisting of mannitol, xylitol, sorbitol and maltitol.

19. The tablet according to claim 17, wherein the cellulosic derivative is microcrystalline cellulose.

20. The tablet according to claim 1, wherein the disintegrating agent is selected from the group consisting of crosslinked sodium carboxymethylcellulose, crospovidone and mixtures thereof.

21. The tablet according to claim 1, wherein the lubricant is magnesium stearate.

22. The tablet according to claim 1, wherein the tablet further comprises one or more excipients selected from the group consisting of a swelling agent, a permeabilising agent, sweeteners, flavourings, cooling agents and colours.

23. The tablet according to claim 2, wherein the tablet is comprised of from 10 to 80 mg of omeprazole or an alkaline salt thereof, and 200-1500 mg of antacid agents.

24. The tablet according to claim 4, wherein the tablet is comprised of omeprazole magnesium in an amount corresponding to 20 mg omeprazole, and antacid agents in an amount of 450 mg.

25. The tablet according to claim 4, wherein the tablet is comprised of omeprazole magnesium in an amount corresponding to 20 mg omeprazole, and antacid agents in an amount of 990 mg.

26. The tablet according to claim 4, wherein the tablet comprises omeprazole magnesium in an amount corresponding to 10 mg omeprazole, and antacid agents in an amount of 495 mg.

27. The tablet according to claim 1, wherein the tablet has a hardness of not less than 15 N.

28. The tablet according to claim 1, wherein the tablet is orodispersible and disintegrates in contact with saliva in the mouth without chewing in less than 60 seconds.

29. The tablet according to claim 28 comprising:
i) enteric coating layered microgranules comprising omeprazole magnesium, wherein the enteric coating layered microgranules are overcoated with the barrier coating comprising Eudragit® E PO (methacrylic copolymer), dibutyl sebacate, sodium lauryl sulphate, magnesium stearate, purified water, and optional ingredients selected from the group consisting of titanium dioxide, hypromellose and talcum;
ii) antacid granules comprising CaCO₃, Mg(OH)₂, mannitol, sorbitol, purified water and optional ingredients selected from the group consisting of crospovidone and silica; and
iii) excipients comprising microcrystalline cellulose, crospovidone, aspartame, flavourings, silica, magnesium stearate and optional cooling agents

30. The tablet according to claim 28, wherein the tablet disintegrates in less than 40 seconds.

31. The tablet according to claim 1, wherein the tablet is chewable.

32. The tablet according to claim 31 comprising:
i) enteric coating layered microgranules comprising omeprazole magnesium, wherein the enteric coating layered microgranules are overcoated with the barrier coating comprising Eudragit® E PO (methacrylic copolymer), dibutyl sebacate, sodium lauryl sulphate, magnesium stearate, purified water, and optional ingredients selected from the group consisting of titanium dioxide, hypromellose and talcum;
ii) antacid granules comprising CaCO₃, Mg(OH)₂, mannitol, sorbitol, purified water and optional ingredients selected from the group consisting of crospovidone and silica; and
iii) excipients comprising microcrystalline cellulose, crospovidone, aspartame, flavourings, silica, magnesium stearate and optional cooling agents

33. A process for the manufacture of a tablet according to any one of claims 1 to 32, comprising the steps:
i) preparing the proton pump inhibitor in the form of enteric coating layered microgranules;
ii) spray coating the enteric coating layered microgranules with the barrier layer;
iii) mixing the thus overcoated enteric coating layered microgranules with the granules of the antacid and a mixture of the disintegrating agent, the diluent agent and the lubricant.

34. The process according to claim 33, wherein the lubricant is sprayed over the surface of the tablet.

35. The process according to claim 33 or 34, wherein the antacid is obtained by dry granulation of CaCO₃ and/or Mg(OH)₂ or Al(OH)₃ with mannitol, followed by wet granulation using a solution of xylitol and/or sorbitol.

36.

37. A method of treatment of gastrointestinal disorders, which comprises administration of a tablet as defined in any of claims 1 to 32 or 38-42 to a patient suffering from gastrointestinal disorders.

38. The tablet according to claim 7, wherein the particle size of the enteric coating layered microgranules is in the range between 100 and 800 microns

39. The tablet according to claim 24, wherein the tablet is comprised of 350 mg CaCO₃ and 100 mg Mg(OH)₂ as the antacid agents.

40. The tablet according to claim 25, wherein the tablet is comprised of 770 mg CaCO₃ and 220 mg Mg(OH)₂ as the antacid agents.

41. The tablet according to claim 26, wherein the tablet is comprised of 385 mg CaCO₃ and 110 mg Mg(OH)₂ as the antacid agents.

42. The tablet according to claim 27, wherein the hardness is between 20 to 70 N.
